(19) United States
(12) Patent Application Publication
(10) Pub. No.: US 2021/0093882 A1
(43) Pub. Date: Apr. 1, 2021

(54) FIXATION IMPLEMENT

(71) Applicant: SHARP KABUSHIKI KAISHA, Sakai City, Osaka (JP)

(72) Inventors: KATSUJI IGUCHI, Sakai City (JP); JUN MORI, Sakai City (JP); HIROYA SATO, Sakai City (JP)

(21) Appl. No.: 16/065,250

(22) PCT Filed: Oct. 4, 2016
(86) PCT No.: PCT/JP2016/079430

§ 371 Date: Jun. 22, 2018

(30) Foreign Application Priority Data
Dec. 24, 2015 (JP) 2015-252577

(51) Int. Cl. A61N 5/06

(52) U.S. Cl. CPC A61N 5/06; A61N 2005/0664; A61N 2005/0626; A61N 2005/0645

(57) ABSTRACT

A burden on a patient in light irradiation therapy is reduced, and treatment that is safe and takes reduced effort is enabled. A hook surface fastener that holds a cooling material of a light irradiating module so that the cooling material is in contact with the light irradiating module is provided on a rear surface of a band and a device pocket that holds a device performing at least one of power supply to and control of the light irradiating module is provided on a front surface of the band so as not to overlap with the hook surface fastener.

### TECHNICAL FIELD

The present invention relates to a fixation implement used for light irradiation therapy.

### BACKGROUND ART

Light irradiation is utilized for various purposes of treatment for diseases such as neonatal jaundice, psoriasis, or acne, alleviation of pain, cosmetic appearance, and the like. Specifically, green light and blue-white light are used for treatment for neonatal jaundice, ultraviolet light is used for treatment for psoriasis, and blue light, red light, and yellow light are used for treatment for acne. In this manner, various light sources are used in accordance with intended use.

For example, in a case of a light source such as an excimer lamp or an arc lamp, therapeutic light is radiated to an affected part arranged at a certain distance from such a light source that is fixed. However, in a case where such light source of a lamp type is used, an irradiation area is large and a part other than an affected part is irradiated with the therapeutic light, and consequently there is a concern about various side effects on a normal part. Thus, some shielding countermeasures need to be taken to prevent the normal part from being irradiated with therapeutic light, and treatment takes time and effort.

For example, when a part of a face where disease has developed is treated, an eye mask (blindfold) to protect the eyes, which are normal parts, is necessary, and, furthermore, a mask which exposes only an affected part of the face is also necessary in order to protect normal parts of the face. Moreover, for the treatment, a patient is required to keep his/her posture without moving for several tens of minutes in a state where his/her body is restrained, and such an experience is not pleasant, even for the treatment.

In a case where an affected part has a curved surface, for example, an affected part is an arm or a foot, an irradiating apparatus of a lamp type may force a patient to take an unnatural posture depending on a part, such as a front part, a rear part, or a side part. In addition, irradiation intensity differs for each position of the affected part in accordance with angle of or distance to the affected part, with respect to the lamp, having the curved part, so that it is difficult to irradiate the entirety of the affected part with uniform therapeutic light in some cases. Further, an apparatus using such a lamp-type light source has many accompanying devices, such as a power source and a cooling device, and is large, and consequently a large space is required for installation and the price of the apparatus increases.

Under such circumstances, some techniques by which light irradiation is able to be performed with an affected part directly covered have been proposed. For example, PTL 1 discloses a light irradiating apparatus having flexibility, in which a large number of LEDs serving as light-emitting light sources are arranged on a flexible substrate and the resultant structure is placed around an affected part to perform light irradiation. PTL 2 discloses a light irradiating apparatus in which an LED serving as a light source is arranged on a flexible substrate and a light-transmitting material is held between an affected part and the LED so that light emitted from the LED is able to be transmitted to the affected part.

### CITATION LIST

### Patent Literature

PTL 1: International Publication No. WO2001014012A1 (published on Mar. 1, 2001)

PTL 2: International Publication No. WO2012023086A1 (published on Feb. 23, 2012)

### SUMMARY OF INVENTION

### Technical Problem

However, the related-art techniques described above have the following problems. In PTL 1, since the light sources are integrated with a fixation implement that fixes the light sources to the affected part, apparatuses of different sizes according to treatment parts such as a trunk, an arm, and a thigh need to be made. In general, a treatment part often has a small area, and in such a case, skin around the treatment part, which has a considerable area, is subjected to light irradiation. Further, the light sources and the fixation implement need to be sterilized for each treatment. As the light sources are able to be cooled by circulation of cooling water, a circulation device for the cooling water is necessary, and as a result apparatus costs increase, in addition to restraining movement of a patient during treatment. Since a power source by which the light sources emit light is also provided separately and is connected to a light irradiating unit by a power cable, it is difficult for the patient to move around as desired during treatment.

In PTL 2, no countermeasure for light source temperature is taken, and no consideration is made to reduce the degree of restraint, due to a power source or a control unit, on the patient.

The invention proposes a fixation implement that reduces the burden on a patient in light irradiation therapy and enables light irradiation therapy that is safe and takes reduced effort.

### Solution to Problem

A fixation implement according to an aspect of the invention is a fixation implement that fixes a light irradiating module for phototherapy to a body and includes at least one band that is placed on the body, a first holding unit that is provided on a rear surface of the band, which is on a body side, and that holds a cooling material of the light irradiating module so that the cooling material is in contact with the light irradiating module, and a second holding unit that is provided on a front surface of the band so as not to overlap with the first holding unit and that holds a device performing at least one of power supply to and control of the light irradiating module.

### Advantageous Effects of Invention

According to an aspect of the invention, the burden on a patient is reduced in light irradiation therapy, enabling light irradiation therapy that is safe and takes reduced effort.

### BRIEF DESCRIPTION OF DRAWINGS

FIG. 1 is a sectional view illustrating a state where a fixation implement according to Embodiment 1 is placed on a body.

FIG. 2 is a plan view illustrating structures of a front surface and a rear surface of the fixation implement according to Embodiment 1.

FIG. 3 is an explanatory view illustrating a structure of a fixation implement according to Embodiment 2.

FIG. 4 is a schematic view illustrating a state where a fixation implement according to Embodiment 3 is placed on a body.

FIG. 5 is a plan view illustrating structures of a front surface and a rear surface of the fixation implement according to Embodiment 3.

FIG. 6 is a schematic view illustrating a state where another fixation implement according to Embodiment 3 is placed on a body.

### DESCRIPTION OF EMBODIMENTS

Hereinafter, embodiments of the invention will be described by exemplifying a case where light irradiation therapy with a light irradiating substrate is performed for a case of skin disease having a relatively small area.

As the light irradiating substrate, for example, a substrate (flexible light irradiating substrate) in which an LED that emits light of a predetermined wavelength is mounted on a resin surface having flexibility to enable lighting is assumed. Note that, normally, medication for treatment is applied to an affected part or taken in advance in many cases. It is assumed that an appropriate distance is maintained between the LED and the affected part in order to uniformly irradiate the affected part with light. However, medication, the light wavelength used for treatment, details of the light irradiating substrate, and so on do not affect the embodiments and are thus not described in detail here.

### Embodiment 1

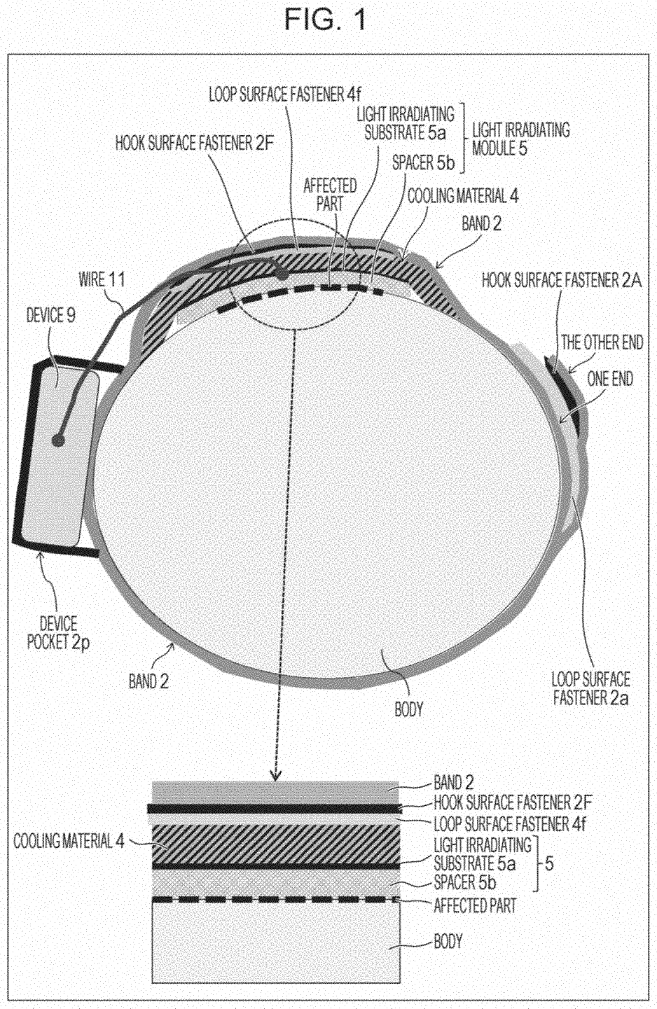

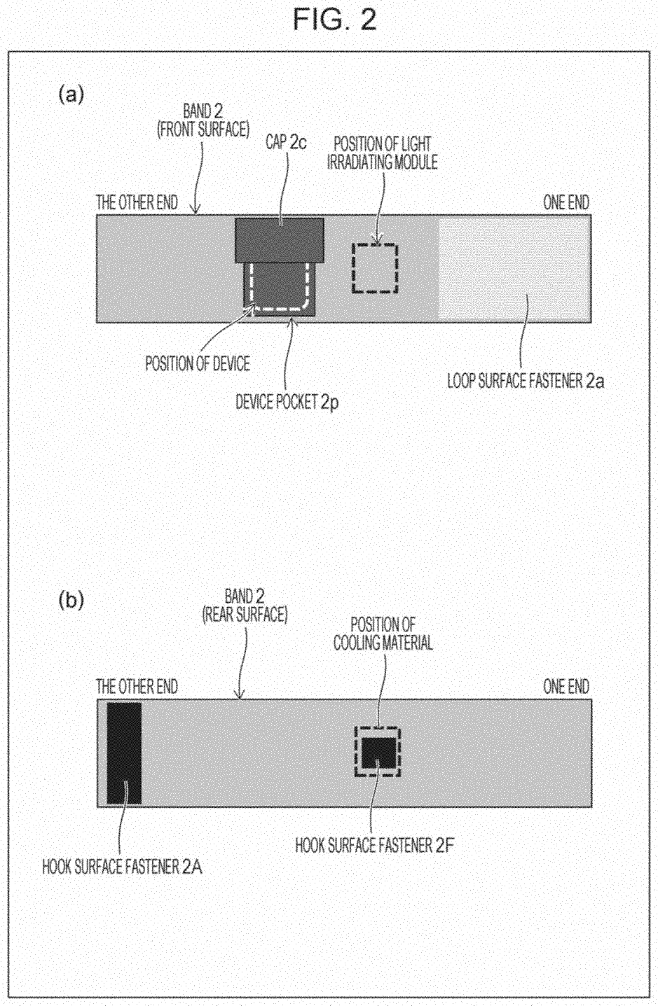

FIG. 1 is a sectional view illustrating a state where a fixation implement of Embodiment 1 is placed on a body to treat an affected part developed in a part of the body, such as a hand, a foot, or a face. FIG. 2 is a plan view illustrating structures of a front surface and a rear surface of the fixation implement of Embodiment 1. In FIG. 2, a state where a band is vertically flipped from a state (front side) of (a) is illustrated as (b) in FIG. 2.

As illustrated in FIGS. 1 and 2, the fixation implement of Embodiment 1 is a fixation implement that fixes a light irradiating module 5 for phototherapy to a body and includes a band 2 that is placed around the body. A hook surface fastener 2F (first holding unit) that holds a cooling material 4 of the light irradiating module so that the cooling material is in contact with the light irradiating module 5 is provided on a rear surface of the band 2, which is on a body side (refer to FIGS. 1 and 2(b)), and a device pocket 2p (second holding unit) that holds a device 9 performing at least one of power supply to and control of a light irradiating substrate 5a is provided on a front surface of the band 2 so as not to overlap with the hook surface fastener 2F (refer to FIGS. 1 and 2(a)).

As illustrated in FIG. 1, the band 2 is stretchable and is placed around the body so as to hold the light irradiating module 5 between the band 2 and the body. The light irradiating module 5 is formed by integrating the light irradiating substrate 5a and a spacer 5b which is arranged on a light irradiation surface side of the light irradiating substrate 5a.

Specifically, the device 9 that is connected to the light irradiating substrate 5a via a wire 11 is stored in the device pocket 2p on the front surface of the band 2, the cooling material 4 is attached to the hook surface fastener 2F on the rear surface of the band 2 by a loop surface fastener 4f which is provided on a front surface (external side) of the cooling material 4, the band 2 is placed around the body so that the spacer 5b is pressed against an affected part and the cooling material 4 is pressed directly against the light irradiating substrate 5a, and a loop surface fastener 2a (one of a pair of surface fasteners) provided on the front surface at one longitudinal end of the band 2 and a hook surface fastener 2A (the other of the pair of surface fasteners) provided on the rear surface at the other longitudinal end of the band 2 are attached to each other so that the band 2 does not become loose.

The device pocket 2p has an access point at an upper part of which a cap 2c (cover) is provided and thus the device pocket 2p is configured to prevent the device 9 from coming out therefrom in response to an action of a patient.

According to the fixation implement of Embodiment 1, it is possible to prevent chipping or movement of the light irradiating module 5 and prevent a failure of the light irradiating substrate 5a and unnecessary stimulation of the patient by heat or electricity, which may be caused when the patient comes into contact with the light irradiating substrate 5a. Such effects are also achieved when the cooling material 4 is not used.

Though the light irradiating substrate 5a and the spacer 5b are collectively called the light irradiating module 5 here, the light irradiating module 5 generally refers to a part extending from a light source to an affected part and does not include a power source that supplies power to the light source. Moreover, medication is usually applied to a surface of the affected part, but is not illustrated here.

The spacer 5b is formed of a material that has flexibility and light-transmitting properties, keeps a constant distance between an affected part and the light irradiating substrate 5a, even when the affected part has a curved surface, and enables the affected part to be irradiated with uniform light. Moreover, the spacer 5b prevents heat generated at the light irradiating substrate 5a from being directly transferred to an affected part.

By covering a rear side (side opposite to the affected part) of the light irradiating substrate 5a with the band 2 that holds the cooling material 4, all of the light irradiating module 5 is fixed to the body. That is, since the cooling material 4 is fixed to the band 2 by the loop surface fastener 4f and the hook surface fastener 2F, when the band 2 is placed around by fitting a position of the cooling material 4 with a position of the light irradiating module 5, rotation, positional shift, or the like of the cooling material 4 is not caused (considering positioning of the band 2 is only required) and the band is able to be easily placed.

When the device pocket 2p is provided near the light irradiating module 5 and the device 9 is connected to the light irradiating substrate 5a by the wire 11, the patient is able to move around almost freely while receiving treatment. An AC adapter or the like is also able to be stored in the device pocket 2p.

The device 9 may be a power source for the light irradiating substrate 5a or a control device for the light irradiating substrate 5a or may be formed by the power source and the control device of the light irradiating substrate 5a being integrated with each other.

Here, a function of the cooling material 4 will be described. In order to perform treatment in a relatively short time, the intensity of light irradiation of the light irradiating substrate needs to be increased, and a certain temperature rise is unavoidable, even when an LED having high efficiency is used. For example, even when light irradiation is performed with an intensity of light irradiation of 100 mW/cm² for about 10 minutes and the power efficiency of an LED is 50%, an energy of 60 J/cm² is generated only in the light irradiating substrate 5a. If a layer of water with a thickness of 5 mm absorbs the energy, temperature rises by about 29 degrees. When it is also considered that light absorbed by the affected part is converted into heat, temperature of the affected part may further rise.

Thus, a certain cooling mechanism is necessary, but when a water cooling mechanism or the like is provided, size and cost of a treatment apparatus increase and movement of the patient is restrained. The easiest cooling method is arranging, on the rear side (side opposite to the affected part) of the light irradiating substrate, a cooling material (cooling means) such as a bag enclosing a heat insulator or water. By using cooling means having reasonable heat capacity, the temperature rise as described above is able to be prevented. In a case where treatment is performed over time with a very low intensity of light irradiation, the cooling means may not be necessary.

In a case where cooling with a cooling material or the like is performed, it is important that the rear side of the light irradiating substrate 5a and the cooling material 4 are in direct contact with each other as illustrated in FIG. 1. For example, in a case where the cooling material is arranged on an opposite side of the band, the band is held between the cooling material and the light irradiating substrate and cooling efficiency is considerably lowered.

In Embodiment 1, as illustrated in FIGS. 1 and 2, by providing the hook surface fastener 2F on the rear surface (affected part side) of the band 2 and the loop surface fastener 4f on the front surface of the cooling material 4, the cooling material 4 is fixed to the band 2 so that the cooling material 4 is in direct contact with the rear side of the light irradiating substrate 5a.

The light irradiating substrate 5a generally has an area larger than that of an affected part in order to perform treatment uniformly for all of the affected part. For efficiently cooling the light irradiating substrate 5a, it is preferable that the cooling material 4 cover a whole of the light irradiating substrate 5a. Moreover, it is preferable that the band 2 cover all of the cooling material 4 to fix the cooling material 4.

It is preferable that a fixation implement of one mode be utilizable for treatment of various parts. Thus, it is desirable that the first holding unit (hook surface fastener 2F) that holds the cooling material 4 and the device pocket 2p that is the second holding unit be as proximate to each other as possible within a range where neither overlaps the other.

In the fixation implement of Embodiment 1, the band 2 is formed by a stretchable bandage material to facilitate fixing of the light irradiating module 5 through pressing against the body.

In FIGS. 1 and 2, both ends of the band 2 are fixed by the pair of surface fasteners, but may be fixed by surgical tape or the like. A removal prevention mechanism for the device is preferably provided in the device pocket 2p, and, as the removal prevention mechanism, a cap with a button that covers the access point of the device pocket is able to be provided, or line fasteners or surface fasteners are able to be provided to the access point of the device pocket, for example.

The fixation implement of Embodiment 1 is entirely formed by consumable members and is able to be handled in a similar manner to a bandage. Thus, the fixation implement is manufactured at a low cost and is also disposable, or is also able to be used a plurality of times through washing and sterilization cycles. A configuration in which the light irradiating module 5 and the fixation implement are completely separate makes it possible to provide the light irradiating module 5 as an inexpensive and versatile product. That is, according to Embodiment 1, it is possible to increase versatility of the fixation implement and the light irradiating module 5 and reduce manufacturing costs.

### Embodiment 2

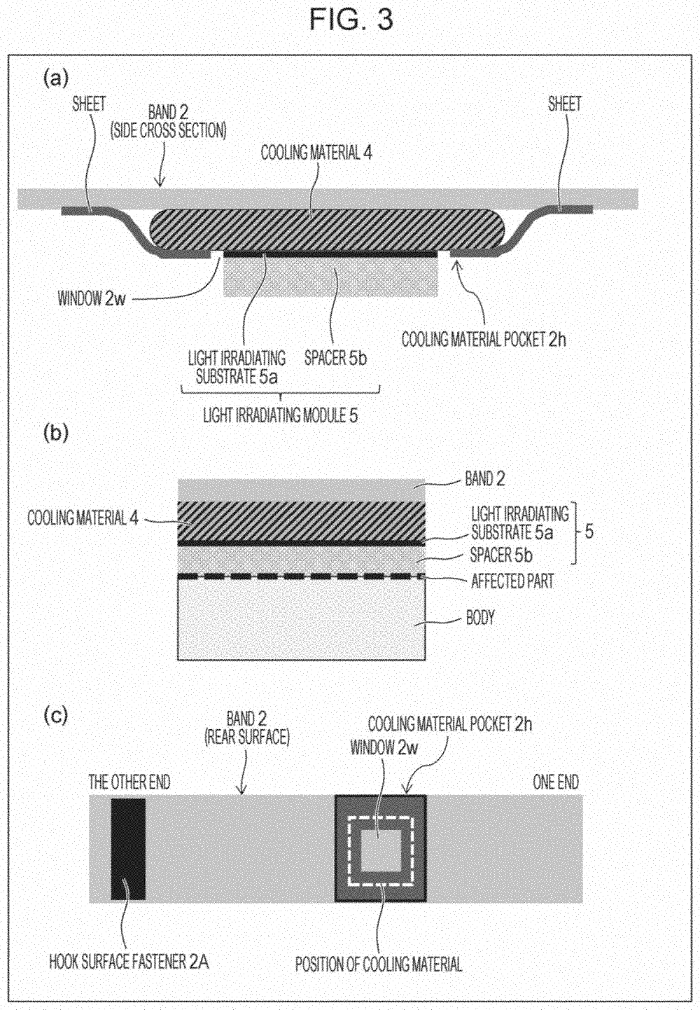

FIG. 3 is an explanatory view illustrating a structure of a fixation implement according to Embodiment 2. The fixation implement of Embodiment 2 differs from the fixation implement of Embodiment 1 in a holding mechanism (a structure of the first holding unit) of the cooling material 4. Specifically, as illustrated in FIGS. 3(a) and (b) that are sectional views of the first holding unit and FIG. 3(c) that is a plan view of the rear surface of the band, a cooling material pocket 2h is provided as the first holding unit on the rear surface (affected part side) of the band 2. The cooling material pocket 2h is formed by a stretchable sheet that has a window 2w (opening) at a center part thereof, and is thus able to store therein the cooling material 4 by inserting the cooling material 4 through the window 2w and is also able to make the cooling material 4 exposed from the window 2w directly in contact with the rear surface of the light irradiating substrate 5a after the storage.

In Embodiment 2, the cooling material pocket 2h is formed by a stretchable cloth (sheet) so that the cooling material 4 is able to be pushed into the cooling material pocket 2h through the window 2w, and thus effort is reduced. That is, a surface fastener does not need to be attached to the cooling material as in Example 1, and it is possible to reduce effort and costs. The cooling material pocket 2h is able to be stitched to the band 2 with a sewing machine or the like or may be attached thereto with an adhesive or double-sided tape.

### Embodiment 3

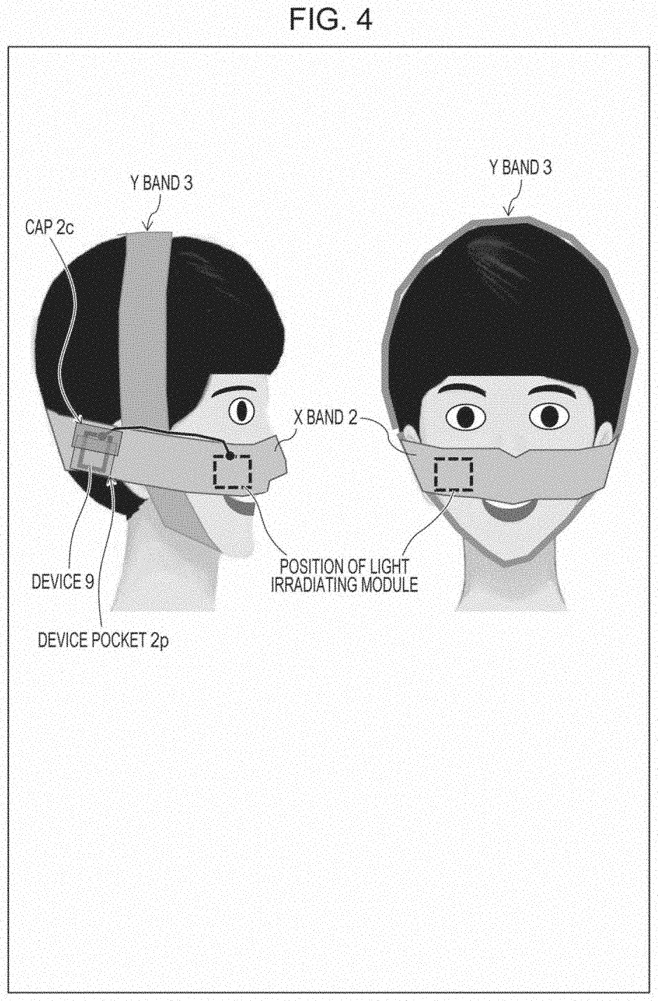

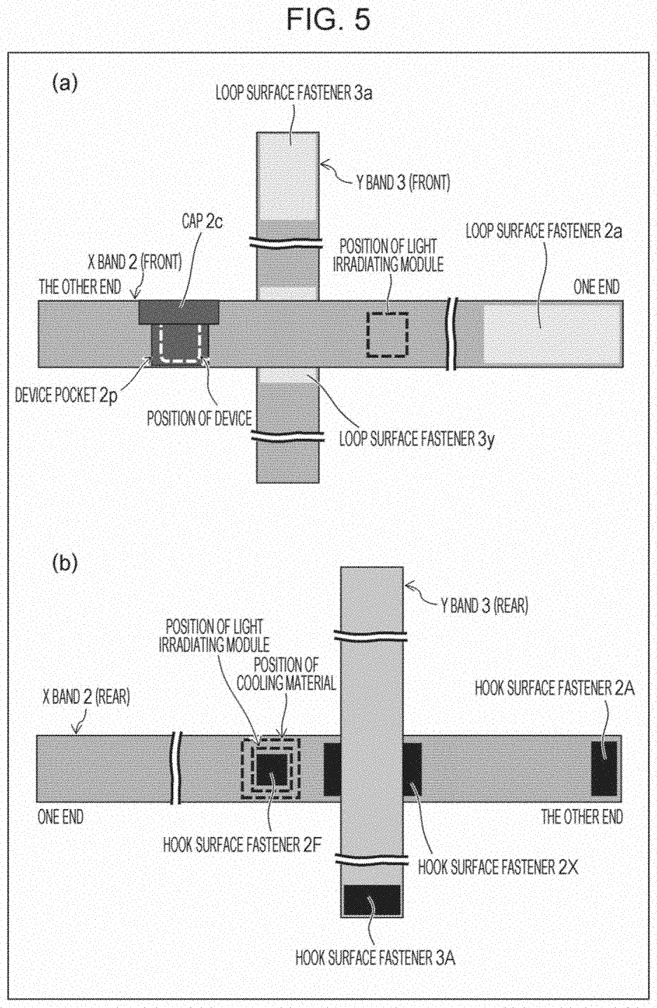

FIG. 4 is a schematic view illustrating a state where a fixation implement according to Embodiment 3 is placed on a body and FIG. 5 is a plan view illustrating structures of a front surface and a rear surface of the fixation implement according to Embodiment 3.

As illustrated in FIGS. 4 and 5, the fixation implement of Embodiment 3 includes an X band 2 that is placed around a head from a back of the head to a face and a Y band 3 that is placed around the head from a top of the head to a jaw. In a case where treatment is performed on a face or head of a human, it may be difficult to perform stable fixation only with one band, and in such a case, by providing, in addition to the X band 2 in a horizontal direction, the different Y band 3 in a direction orthogonal thereto, stability is able to be enhanced. In FIG. 5, a state where the X band and the Y band are horizontally flipped from a state (front side) of (a) is illustrated as (b) in FIG. 5.

As illustrated in FIG. 5(a), on a front surface of the X band 2, the device pocket 2p (second holding unit) that holds the device 9 performing at least one of power supply to and control of the light irradiating substrate is provided so that the device pocket 2p does not overlap with the hook surface fastener 2F, and further, the loop surface fastener 2a used for fixation of the X band itself is provided at one longitudinal end.

As illustrated in FIG. 5(b), on a rear surface of the X band 2, which in on a body side, the hook surface fastener 2F (first holding unit) that holds the cooling material 4 of the light irradiating module so that the cooling material 4 is in contact with the light irradiating module, the hook surface fastener 2A that is arranged at the other longitudinal end, and a hook surface fastener 2X that is provided between the hook surface fastener 2F and the hook surface fastener 2A are provided. The hook surface fastener 2A is used for fixation of the X band itself and the hook surface fastener 2X is used for fixation between the X band and the Y band 3.

In addition, as illustrated in FIGS. 5(a) and (b), a loop surface fastener 3a that is used for fixation of the Y band itself and a loop surface fastener 3y that is used for fixation between the Y band 3 and the X band 2 are provided on a front surface of the Y band 3, and a hook surface fastener 3A that is used for fixation of the Y band itself is provided on a rear surface of the Y band 3, which in on a body side.

In Embodiment 3, since the X band 2 and the Y band 3 are fixed by the hook surface fastener 2X and the loop surface fastener 3y, an attachment angle of the Y band 3 relative to the X band 2 is able to be changed and mutual fixing positions are able to be changed, so that a fixing method is easily optimized in accordance with an affected part.

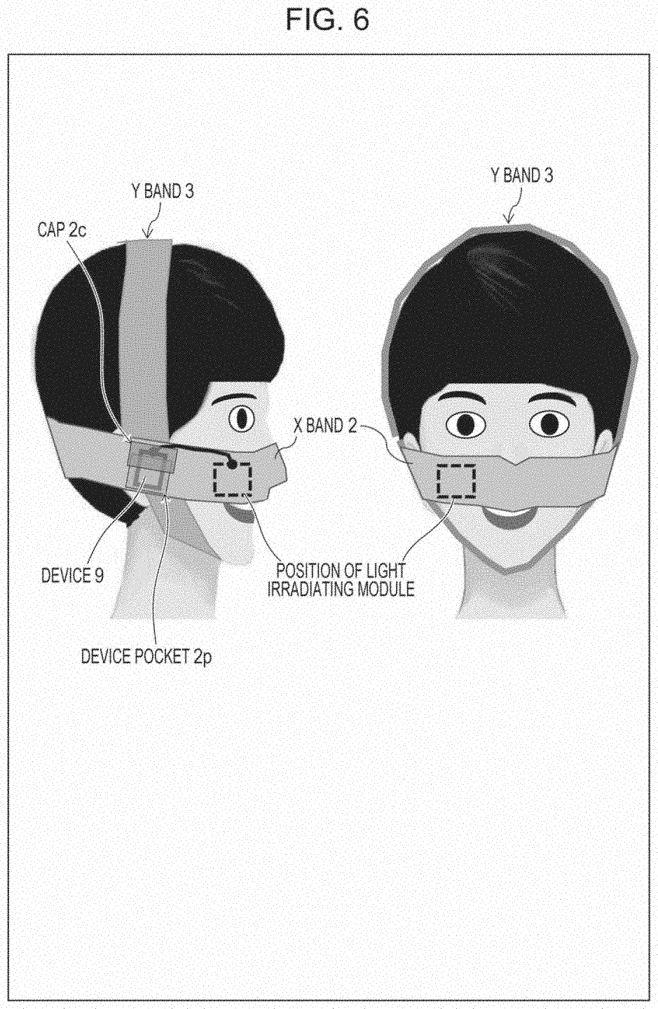

In Embodiment 3, the device pocket 2p (second holding unit) of the X band 2 may be provided also in a part crossing the Y band 3 as illustrated in FIG. 6. That is, the device pocket 2p is provided on the front surface of the X band 2 so that the device pocket 2p overlaps with the hook surface fastener 2X on the rear surface. As a result, even when the device 9 is heavy to some extent, the X band 2 is prevented from being shifted downward and the device 9 is able to be stably held with respect to a head.

### CONCLUSION

A fixation implement according to an aspect 1 of the invention is a fixation implement that fixes a light irradiating module for phototherapy to a body, and includes at least one band that is placed on the body, a first holding unit that is provided on a rear surface of the band, which is on a body side, and that holds a cooling material of the light irradiating module so that the cooling material is in contact with the light irradiating module, and a second holding unit that is provided on a front surface of the band so as not to overlap with the first holding unit and that holds a device performing at least one of power supply to and control of the light irradiating module.

According to the aforementioned configuration, a burden on a patient in light irradiation therapy is reduced and light irradiation therapy that is safe and takes reduced effort is enabled.

The fixation implement according to an aspect 2 of the invention is configured, in the aspect 1, so that the band is placed on the body so as to hold the light irradiating module between the band and the body.

The fixation implement according to an aspect 3 of the invention is configured, in the aspect 2, so that the light irradiating module is formed by integrating a spacer and a light irradiating substrate, and when the band is placed on the body, the spacer comes into contact with the body and the light irradiating substrate comes into contact with the cooling material.

The fixation implement according to an aspect 4 of the invention is configured, in the aspect 1 or 2, so that the band is stretchable.

The fixation implement according to an aspect 5 of the invention is configured, in any of the aspects 1 to 4, so that one of a pair of surface fasteners is provided at one end of the band and the other of the pair of surface fasteners is provided at the other end of the band.

In the fixation implement according to an aspect 6 of the invention, in any of the aspects 1 to 5, the first holding unit is formed by any one of the pair of surface fasteners.

In the fixation implement according to an aspect 7 of the invention, in any of the aspects 1 to 5, the first holding unit is constituted by a stretchable pocket that has a window.

In the fixation implement according to an aspect 8 of the invention, in any of the aspects 1 to 7, the second holding unit is constituted by a pocket with a cover.

The fixation implement according to an aspect 9 of the invention is configured to, in any of the aspects 1 to 8, include a different band that is placed on the body so as to cross the band.

The fixation implement according to an aspect 10 of the invention is configured, in any of the aspects 1 to 8, so that the second holding unit is provided in a part crossing the different band.

The invention is not limited to the embodiments described above, and an embodiment achieved by appropriately combining technical means disclosed in each of different embodiments is also encompassed in the technical scope of the invention. Further, by combining the technical means disclosed in each of the embodiments, a new technical feature may be formed.

### REFERENCE SIGNS LIST

2 band, X band
2F hook surface fastener (first holding unit)
2p device pocket (second holding unit)
2A hook surface fastener
2a loop surface fastener
2X hook surface fastener
2h cooling material pocket (first holding unit)
3 Y band
3y loop surface fastener
4 cooling material
5 light irradiating module
5a light irradiating substrate
5b spacer

1. A fixation implement that fixes a light irradiating module for phototherapy to a body, the fixation implement comprising
at least one band that is placed on the body;
a first holding unit that is provided on a rear surface of the band, which is on a body side, and that holds a cooling material of the light irradiating module so that the cooling material is in contact with the light irradiating module; and
a second holding unit that is provided on a front surface of the band so as not to overlap with the first holding unit and that holds a device performing at least one of power supply to and control of the light irradiating module.

2. The fixation implement according to claim 1, wherein the band is stretchable.

3. The fixation implement according to claim 1, wherein one of a pair of surface fasteners is provided at one end of the band and the other of the pair of surface fasteners is provided at the other end of the band.

4. The fixation implement according to claim 1, wherein the first holding unit is formed by any one of the pair of surface fasteners.

5. The fixation implement according to claim 1, wherein the first holding unit is constituted by a stretchable pocket that has a window.

6. A fixation implement that fixes a light irradiating module for phototherapy to a body, the light irradiating module and the fixation implement being constituted to be separate from each other, the fixation implement comprising:
at least one band that is placed on the body;
a first holding unit that is provided on a rear surface of the band, which is on a body side, and that holds a cooling material of the light irradiating module so that the cooling material is in direct contact with the light irradiating module; and
a second holding unit that is provided on a front surface of the band so as not to overlap with the first holding unit and that holds a device performing at least one of power supply to and control of the light irradiating module.
